(19) United States
(12) Patent Application Publication
(10) Pub. No.: US 2006/0281960 A1
(43) Pub. Date: Dec. 14, 2006

(54) Process and installation for separating compounds, including at least one n-paraffin, in a hydrocarbon feedstock

(76) Inventors: Elsa Jolimaitre, Lyon (FR); Laurent Bournay, Chaussan (FR); Arnaud Baudot, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/444,527

(22) Filed: Jun. 1, 2006

(30) Foreign Application Priority Data
Jun. 3, 2005 (FR) 05/51.492

(51) Int. Cl. C07C 7/144

(52) U.S. Cl. 585/818

(57) ABSTRACT

Process for separating one or more compound(s), including at least one n-paraffin, in a hydrocarbon feedstock, in which: A stream, or a portion of a stream of said feedstock, is passed successively and in any order into at least one distillation column and at least one membrane separation unit comprising a selective membrane with regard to at least one n-paraffin; and One or more stream(s) is (are) recovered from the column and the membrane separation unit, each of said streams being enriched respectively with one of said compounds, and at least one of said streams being enriched with at least one n-paraffin. Installation for the implementation of this process. The invention relates in particular to a process and an installation that allow the fractionation of a petroleum fraction that contains hydrocarbon molecules with close boiling points.

### FIELD OF THE INVENTION

The invention relates to a process and an installation for separating compounds, including at least one n-paraffin, from a hydrocarbon feedstock.

The invention relates in particular to a process and an installation that allow the fractionation of a petroleum fraction containing hydrocarbon molecules with close boiling points.

Inside of a given fraction, different chemical families may be encountered, and inside of the same family, elements of the same chemical formula but of different geometric configurations referred to as isomers may be encountered.

The invention is therefore suitable for the separation of these isomers, but more particularly for the separation of at least one normal paraffin inside of a hydrocarbon feedstock or any petroleum fraction, in particular having the same number of carbon atoms as the normal paraffin that is being considered.

The invention can be applied particularly advantageously in the separation of molecules, in particular normal paraffins contained in hydrocarbon fractions with a given number of carbon atoms ranging from 4 to 16, and more particularly with a number of carbon atoms ranging from 4 to 10, for example the C4, C5, C6 or C7 fractions.

The process or the installation according to the invention can be applied or integrated in any process or installation of refining or petrochemistry; in particular the installation of the invention is to be considered as a module that can be used in various processes of refining or petrochemistry.

### Prior Art.

The separation of molecules with a close boiling point is much used in refinery processes and applies to various petroleum fractions.

The molecules in question are most often paraffins with different degrees of branching, naphthenic compounds, aromatic compounds and olefinic compounds.

Thus, the separation of normal butane from isobutane, which may or may not be coupled to an isomerization, is essential for the synthesis of the isooctane or 2,2,4 trimethylpentane.

Likewise, the separation of the normal paraffins and optionally mono-branched paraffins from the C5-C7 fraction is often carried out in a refinery with the objective of increasing the research octane number (or “RON” in English) of said gasoline fraction.

It is also possible to cite the separation of the cyclohexane of the C6 fraction or the methylcyclohexane of the C7 fraction so as to upgrade them as solvents.

Generally, the separation of the above-mentioned molecules is carried out by distillation.

However, these molecules having very close boiling points, the separation by distillation is difficult and expensive and is reflected by columns consisting of several tens of plates with reflux rates close to 10 and sometimes more than 10.

The final purities are not always satisfactory.

Consequently, a process that makes it possible to carry out this type of separation at a low energy cost and with a better purity of the effluents than the distillation processes would have an obvious industrial advantage.

The processes for separation by adsorption are also commonly used for this type of separation.

Thus, the patents U.S. Pat. No. 4,608,061 and U.S. Pat. No. 6,022,398 relate to the separation of the normal butane from isobutane by pressure-modulated adsorption (“pressure swing adsorption” or “PSA” in English).

This same technique is claimed in the patent application WO-A-03/040266 for the separation of normal paraffins of the C5-C10 fraction.

The patents U.S.-B1-6,353,144 and U.S. Pat. No. 6,069,289 claim the separation by adsorption of paraffins with different degrees of branching in simulated countercurrent-type processes (abbreviation “CCS”).

These processes for separation by adsorption, in particular in simulated countercurrent, make it possible to obtain a high purity of different products but also exhibit a certain number of drawbacks, including a great complexity of implementation and a high operating cost.

Furthermore, the patents U.S. Pat. No. 5,146,037 and U.S. Pat No. 5,043,525 describe the combination of a distillation column and a separation by adsorption on the C5-C6 fraction. The distillation column makes it possible to carry out a first separation of the effluent so as to reduce the feedstock to be treated by adsorption. Reducing the sieve mass to be used in the adsorption unit and increasing the final purity are thus achieved. This type of process nevertheless exhibits a significant drawback: the distillation and the separation by adsorption are both very energy-intensive. The use of these two coupled techniques is not favorable to a reduction in the operating costs of the separation.

The absorption is another separation technique used to treat the petroleum fractions.

Thus, the patent U.S. Pat. No. 3,169,998 proposes carrying out the separation of C7 molecules (normal C7 and methylcyclohexane) by absorption in a selective solvent with the cyclic isomer (methylcyclohexane).

Likewise, the patent U.S. Pat. No. 5,107,059 discloses a technique of normal separation/isoparaffins by solvent, whereby the contact with the solvent is carried out via a non-selective membrane.

The selectivity of the solvents described in these patents is not important enough, however, that this type of process is truly competitive, even in coupling with a distillation as described in Patent Application WO-A-02/22528.

The separation of C4-C10 molecules is also conceivable by membrane, a technique that is known for exhibiting numerous advantages such as: modularity, low energy consumption compared to a conventional distillation, low maintenance costs (because of the absence of moving elements), capability of carrying out difficult separations, in particular the separation of mixtures of molecules by the effect of size exclusion.

The MFI-type zeolitic membranes are most commonly cited in the literature, but other membrane materials can also be used to carry out this type of separation:
The patent U.S. Pat. No. 5,914,434 has, for example, a carbon membrane for separating the linear alkanes from the branched alkanes;
The patent U.S. Pat. No. 1-2004/0173529 proposes carrying out the separation of the normal butane from isobutane with organic membranes made of a polymer material.

The membrane processes generally do not make it possible to obtain high purities because the membrane surfaces that are to be used increase very significantly with the desired purity; in particular they increase also in a manner proportional to the flow rate of the feedstock to be treated and can thereby become very significant in the case of separation of the petroleum fractions.

Consequently, the patent U.S. Pat. No. 1-2003/0233934 proposes improving the purity of the products that are obtained from the membrane separation by adding a dephlegmator in the permeate flow. The dephlegmation processes, coupling transfer of material and heat, are expensive and very difficult to use relative to the distillation columns. According to the terminology of one skilled in the art in this field of the art, dephlegmator is defined as a group of condensers that are generally located downstream from a membrane separation unit and that have as their object to facilitate the separation of different compounds contained in the permeate.

This is not an integration per se with the membrane separation unit, besides the fact that the different condensers can in no case be considered as distillation columns.

In view of the preceding, there therefore exists a need for a process—and an installation—for separating n-paraffms from a hydrocarbon feedstock that is simple, reliable, easy to use, and flexible, which involves small investments and low operating costs, whose energy consumption is small, and which makes it possible to obtain the desired compounds, and in particular the n-paraffins, with a very high purity.

The object of this invention is to provide a process—and an installation—for separating compounds and in particular n-paraffins from a feedstock of hydrocarbons that meet, among other things, these needs.

The object of this invention is also to provide a process—and an installation—to separate compounds and in particular normal paraffins from a hydrocarbon feedstock that does not exhibit drawbacks, limitations, defects and disadvantages of the processes—and installations—of the prior art, such as the distillation processes or else the adsorption processes, the absorption processes, or else the membrane processes, and which resolves the problems posed by the processes—and installations—of the prior art.

### Summary Description of the Invention

This object and others as well are attained, according to the invention, by a process for separating one or more compounds, including at least one n-paraffin, in a hydrocarbon feedstock, in which:
A stream, or a portion of a stream from said feedstock, is passed successively and in any order into at least one distillation column and at least one membrane separation unit comprising a selective membrane with regard to at least one n-paraffin; and
One or more stream(s) is (are) recovered from the column and the membrane separation unit, each of said streams being enriched respectively with one of said compounds, and at least one of said streams being enriched with n-paraffin.

It is specified that a membrane separation unit can comprise one or more membrane separation modules.

More specifically, the invention describes a process for separating one or more compound(s), including at least one n-paraffin, in a hydrocarbon feedstock, in which:
A stream or a portion of a stream of said feedstock is passed into at least one distillation column in which at least one incoming stream enters and from which at least one outgoing stream exits, and in at least one membrane separation unit comprising a membrane that is selective with regard to at least one n-paraffin, a membrane separation unit being placed in the stream or streams entering and exiting from the distillation column;
One or more streams are recovered from the column and from the membrane separation unit or units, each of said streams being enriched respectively with one of said compounds, and at least one of said streams being enriched with at least one n-paraffin.

The stream of the feedstock can be treated in its entirety by the process of the invention, namely by at least one distillation column and at least one membrane separation unit, or else only a portion of the stream can be treated by the process according to the invention; the other portion of the stream of the feedstock then being treated only by a portion of this process: i.e., this portion is treated only by either at least one distillation column or at least one membrane separation unit and not by the combination of a distillation column and a membrane separation unit.

By “enriched,” it is understood that in the recovered stream that contains it, the molar concentration of the “separated” compound is greater than the one that it had in the feedstock stream prior to all treatment by the process according to the invention.

For a membrane, an enrichment factor is defined with regard to a molecule by the ratio between the molar concentration of the compound in the permeate to the molar concentration of the compound in the feedstock; if this ratio is greater than 1, there is enrichment and the membrane is selective with regard to this molecule.

Regarding the n-paraffins, the process according to the invention can be used for recovering the most possible n-paraffin(s) from a feedstock. Preferably, said n-paraffin enriched stream that is recovered has a majority content by mass, namely of more than 50%, of n-paraffin(s), and preferably also said recovered stream has a content by mass that is greater than 75% or even 90% by mass of n-paraffins. This stream can then be sent to a unit for transformation of n-paraffins, for example an isomerization.

It is even possible, thanks to the process according to the invention, to recover pure or virtually pure streams (i.e., whose content by mass is close to or equal to 100% of separated compound) from each of the compounds, for example, n-paraffins that it is desired to separate from the feedstock.

The process according to the invention can also be used to purify a hydrocarbon feedstock by removing n-paraffms that then constitute undesirable products.

In this case, the stream, resulting flow, is enriched with n-paraffins but their concentration is advantageously less than or equal to 50%, and even less than 30%. This type of process can be used, for example, to purify the gasolines, such as the C5-C6 fraction before treatment to increase the octane number.

The drawn-off stream is recovered or recycled in its entirety or partially to further enrich it; most often, the recycling is carried out to the distillation column(s).

The process according to the invention can be defined as a process for separating one or more compound(s), including the n-paraffins, that are found in a hydrocarbon feedstock, in which are used the coupling, the combination of at least one distillation unit and at least one membrane separation unit.

The combination, the coupling, in such a process for separating at least one distillation column and at least one membrane separation unit, is neither described nor suggested in the prior art as had been disclosed in particular above.

The membrane(s) used in the membrane separation unit is (are) characterized by its (their) ability to separate the normal paraffins from other molecules contained in said feedstock or fraction.

In other words, the membrane(s) used in the membrane separation unit is (are) selective with regard to at least one n-paraffin; the membrane can be selective with regard to all the n-paraffins or else only with regard to certain of them or a single one among them.

By “selective” membrane with regard to a given molecule such as a normal paraffin, it is generally understood that this molecule passes through the membrane and is found in the permeate while the other molecules of the feedstock do not pass through or almost do not pass through the membrane, are retained by the latter and are found in the retentate.

The process according to the invention does not exhibit the defects of the processes of the prior art, in particular processes that use only one distillation column, and it provides a solution to the problems of the processes of the prior art.

The process according to the invention is simple, reliable, certain, easy to use, offers the advantage of carrying out the separation of n-paraffins (at least) with minimum financial and energy expenditure and/or with a higher purity, in particular a separation by distillation alone. It also makes it possible to improve considerably the flexibility of a separation system by simple distillation.

The coupling between the distillation column and the membrane separation unit that is selective for normal paraffins produces a veritable synergy that will be reflected as a whole, among other things, by an increase in the treatment ability of the distillation column and/or a savings on the operating costs (whereby a portion of the separation is carried out by the membrane, this separation being energy-efficient) and/or an increase of the purity of the final products (with isocharge).

No reading of the prior art would suggest that such a totally unexpected synergetic effect could be obtained by coupling, combining, at least one distillation column and at least one membrane separation unit.

In addition, the use of a membrane separation unit leads to an improvement of the flexibility of the separation process according to the invention, which is reflected by a clearly improved ability to deal with the market changes of said process, such as the changes in nature of the feedstock and/or the flow rate of said feedstock.

Furthermore, it is always possible to add a membrane separation module to one or more membrane separation units to increase, for example, the capacity, whereas when the column is in operation, if the maximum feedstock flow rate is reached, it is not possible to increase the capacity of the installation.

According to an embodiment of the invention, the stream of the feedstock or a portion of this stream first is passed into a membrane separation unit then into a distillation column, which means that the membrane separation unit is placed in a stream entering said distillation column or else that the membrane separation unit is “upstream” from the distillation column; in a conventional way, the membrane separation unit separates the feedstock stream or a portion of this stream into a retentate stream and a permeate stream.

In the case where only a portion of the feedstock stream is passed into the membrane separation unit then into the column, the other portion of the feedstock stream is sent directly into the column.

The retentate stream and the permeate stream can both be introduced into the distillation column.

Or else one of the streams from among the permeate stream and the retentate stream is introduced into the column and the other of these streams is recovered.

When the retentate stream and the permeate stream are both introduced into the distillation column, they are separate points of the column therein.

Advantageously, the permeate stream can be introduced into the distillation column at a point where the composition of an inside stream that travels through the column is close to the composition of the permeate stream, and/or the retentate stream can be introduced into the distillation column at a point where the composition of the inside stream that travels through the distillation column is close to the composition of the retentate stream.

Generally, a top stream, a bottom or base stream, and optionally at least one lateral stream exit are drawn off from the distillation column and recovered or recycled completely or partially; these recovered or recycled streams constitute the streams exiting from the distillation column.

According to another embodiment of the invention, the stream of the feedstock (or a portion of this stream), which then constitutes the sole stream entering the column, is passed first into a distillation column from which exits a top stream of the column, a base stream of the column, and optionally at least one lateral stream of the column, which means that a membrane separation unit is placed in a stream exiting from said distillation column, or else a membrane separation unit is placed “downstream” from said distillation column. At least one among the top stream, the base stream and the possible lateral stream or streams of the column is then sent into a membrane separation unit.

It thus is possible to send the top stream of the column into a membrane separation unit that separates the top stream into a retentate stream and a permeate stream.

Or else it is possible to send the base stream of the column into a membrane separation unit that separates the base stream into a retentate stream and a permeate stream.

Or else it is possible to send at least one lateral stream of the column into a membrane separation unit that separates the lateral stream into a retentate stream and a permeate stream.

The permeate stream and/or the retentate stream can be recovered or recycled completely or partially.

Thus, the permeate stream and the retentate stream can both be recovered completely or partially.

Or, for example, one of the streams from among the retentate stream and the permeate stream is at least partially recycled.

Or the permeate and retentate streams are both partially recycled

The permeate stream and/or the retentate stream can be sent upstream or downstream from the membrane separation unit or can be directly recovered if it is considered that, for example, one or the other of these streams contains a compound that it is desired to separate in an adequate concentration.

Or the permeate stream and/or the retentate stream can be recycled to, introduced again into, the distillation column.

The retentate stream and the permeate stream preferably can both be introduced again into the distillation column at separate points.

The permeate stream can be introduced again into the distillation column at a point where the composition of the inside stream that travels through the column is close to the composition of the permeate stream, and/or the retentate stream can be introduced again into the distillation column at a point where the composition of the inside stream that travels through the distillation column is close to the composition of the retentate stream.

In a general way, and regardless of the embodiment, at least one of the streams drawn off at the end of the process (retentate, permeate, of any of the membrane separation units, top stream, lateral stream, base stream, any of the distillation columns, etc . . . ) can be recycled completely or partially to the distillation column or columns and/or to the membrane separation unit or units and/or in the feedstock; thus, at least one of the streams obtained from the membrane separation unit(s) can be recycled completely or partially in said separation.

It is possible to pass the feedstock stream into one or more, for example 2, membrane separation unit(s), each comprising 1 or more membrane modules, according to various arrangements that are well known to one skilled in the art.

Thus, if the feedstock stream (or a portion of the latter) passes into two membrane separation units, one of these units can be placed in a stream entering the distillation column, upstream from the column, and the other can be placed downstream from the column in a stream exiting the distillation column, selected from among, for example, the top stream, the base or bottom stream and a lateral stream.

The treated hydrocarbon feedstock can be of any type.

The hydrocarbon feedstock can be selected from among the petroleum fractions Cn where n is an integer from 4 to 16, preferably 4 to 10, and more preferably 4 to 7, whereby n can take on all the values from 4 to 16.

The hydrocarbon feedstock can also be selected from among the petroleum fractions Cn-Cn+1, where n is an integer from 4 to 16, preferably 4 to 10, and more preferably 4 to 7, whereby n can take on all values from 4 to 16.

Generally, the compound or compounds to be separated can be hydrocarbon compounds that exhibit close boiling points.

Said compound or compounds to be separated can be selected from among—in addition to the n-paraffins—branched paraffins, naphthenic compounds, aromatic compounds and olefinic compounds.

Advantageously, said feedstock can contain said normal paraffin and hydrocarbon compounds that have the same number of carbon atoms as said normal paraffin.

Advantageously, with the process according to the invention, it is possible to separate the normal butane and the isobutane that are contained in a hydrocarbon feedstock from hydrocarbons.

Advantageously, also, with the process according to the invention, it is possible to separate the isopentane and the normal pentane that are contained in a hydrocarbon feedstock.

The process according to the invention can also make possible the fractionation of a C6 hydrocarbon fraction into three streams, respectively enriched with naphthenic compounds, normal hexane, and branched paraffins.

The process according to the invention can also carry out the fractionation of a C6 hydrocarbon fraction into four streams, respectively enriched with naphthenic compounds, normal hexane, monobranched paraffins, and dibranched paraffins.

The process according to the invention can carry out the fractionation of a C7 hydrocarbon fraction into three streams, respectively enriched with naphthenic compounds, normal heptane, and branched paraffins.

Finally, the process according to the invention can make possible the fractionation of a C7 hydrocarbon fraction into four streams, respectively enriched with naphthenic compounds, normal heptane, monobranched paraffins, and dibranched paraffins.

The invention also relates to an installation for separating one or more compound(s), including at least one n-paraffin, into a hydrocarbon feedstock, comprising:
At least one distillation column;
At least one membrane separation unit comprising at least one selective membrane with regard to at least one n-paraffin;
Means for feeding said installation with a stream of said hydrocarbon feedstock;
Means for ensuring fluid communication for the distillation column or columns and the membrane separation unit or units and passing said stream of said hydrocarbon feedstock or a portion of said stream successively and in any order into the column or columns and the membrane separation unit or units, and;
Means for recovering one or more stream(s) from the column(s) and the membrane separation unit or units, each of said streams being enriched respectively with one of said compounds, and at least one of said streams being enriched with at least one n-paraffin.

Generally, the installation according to the invention comprises a single distillation column (or optionally several columns that are connected in series) and one or more membrane separation units.

According to a first primary embodiment of the installation of the invention, a membrane separation unit is placed upstream from the distillation column in at least one of the streams entering the distillation column.

According to a second primary embodiment of the installation of the invention, a membrane separation unit is placed downstream from the distillation column in at least one of the streams exiting from the distillation column.

In the first primary embodiment of the installation according to the invention, it generally comprises means for sending the feedstock stream or a portion of the latter into said membrane separation unit, means for sending the retentate and/or the permeate of the membrane separation unit into said distillation column, and/or means for recovering the retentate and/or the permeate from said membrane separation unit, and means for drawing off and optionally recovering a top stream, a base stream, and optionally at least one lateral stream from said column.

In the second primary embodiment of the installation according to the invention, it can comprise means for sending the feedstock stream or a portion of the latter into the distillation column, means for sending a top stream of the distillation column into the membrane separation unit, and means for drawing off and optionally recovering a base stream from the distillation column and optionally at least one lateral stream from said distillation column.

Or else still in the second primary embodiment according to the invention, the installation can comprise means for sending the feedstock stream or a portion of the latter into the distillation column, means for sending at least one lateral stream of the distillation column into the membrane separation unit, and means for drawing off and optionally recovering a base stream of the distillation column and a top stream of the distillation column.

Or else also in the second primary embodiment according to the invention, the installation can comprise means for sending the feedstock stream or a portion of the latter into the distillation column, means for sending a base stream of the distillation column into the membrane separation unit, and means for drawing off and optionally recovering a top stream from the column and optionally at least one lateral stream from the distillation column.

In the second primary embodiment according to the invention, the installation generally comprises means for recovering the retentate and/or the permeate from the membrane separation unit and/or means for sending the retentate and/or the permeate into the distillation column.

The installation according to the invention can comprise several membrane separation units, for example the installation according to the invention can comprise two membrane separation units, one being placed upstream from the distillation column and the other being placed downstream from the distillation column.

The installation according to the invention can comprise means for recycling any of the streams obtained from the distillation column or columns and the membrane separation unit or units to the distillation column or columns and/or to the membrane separation unit or units and/or to the feedstock stream.

The membrane of the membrane separation unit can be made of a material that is selected from among the inorganic materials, the organic materials, and the composite materials that comprise several different materials that are selected from among the organic and inorganic materials.

Thus, the membrane can be made of an inorganic material, optionally deposited on a porous substrate, for example made of mineral oxide, such as alumina.

In particular, the membrane can be made of a molecular sieve that optionally is deposited on a porous substrate.

This molecular sieve can be in particular a zeolite, selected from among, for example, the MFI-type zeolites and the LTA-type zeolites.

The membrane can also be made of an organic material, which can be selected from among, for example, the polyimides, the polyamides and the polysulfones.

The membrane separation unit can adopt various modes of operation; it can operate, for example, in a vapor permeation mode, a pervaporation mode, or in a hyperfiltration mode.

Finally, the distillation column can be a partition column.

The invention will now be described in a detailed manner in the following description of embodiments of the installation and the process of the invention given in relation to the attached drawings in which:

### BRIEF DESCRIPTION OF THE DRAWINGS

FIG. 1 is a diagrammatic cutaway view of an installation according to the invention in a first embodiment in which the membrane separation unit is found in a pretreatment configuration of the feedstock feeding the column.

FIG. 2 is a diagrammatic cutaway view of an installation according to the invention in a second embodiment in which the membrane separation unit is found in a post-treatment configuration of the effluent of the bottom of the column.

FIG. 3 is a diagrammatic cutaway view of an installation according to the invention in a third embodiment in which the membrane separation unit is found in a post-treatment configuration of the top effluent of the column.

FIG. 4 is a diagrammatic cutaway view of an installation according to the invention in a fourth embodiment in which the membrane separation unit is found in a treatment configuration of a lateral flow or stream obtained from the column.

### DETAILED DESCRIPTION OF THE INVENTION

Several embodiments of the process and the installation according to the invention are described in detail below, each of these embodiments corresponding to a configuration of the membrane separation unit relative to the distillation column.

Actually, the membrane separation unit can be placed in different locations relative to the distillation column. In particular, it can be placed either in the input stream of the column or in any output stream such as the top stream, or else in the base or bottom stream, or else in any lateral stream of said column.

It is quite obvious that other embodiments and configurations can be considered, for example embodiments in which several membrane separation units are used, each of these units being placed in any of the incoming or outgoing streams (top, bottom, lateral) of the distillation column.

It is also possible to imagine embodiments that use several distillation columns combined with one or more membrane separation units.

All of these embodiments and configurations, whether or not they are described below, are linked to one another by a common inventive concept that is the combination, the coupling, of at least one distillation column with at least one membrane separation unit.

In the following description, primarily the treatment of the entire feedstock stream in the process or the installation of the invention is described; it is quite obvious that only a portion of the feedstock stream can be treated in the installation or the process of the invention, the other portion then being treated only in a portion of the process or installation.

In addition, if the following description is made, for the sake of convenience rather than in relation to the installation according to the invention, it is obvious that it also applies to the process according to the invention.

The invention applies in particular to the treatment of any petroleum fraction that is characterized by a given number of carbon atoms Cn, or an interval Cn Cn+1 relating to two successive values of the number of carbon atoms, such as, for example, the C5/C6 or C4/C5 fractions. To designate all of the petroleum fractions covered by the invention, mention will be made of the Cn Cn+1-type petroleum fraction, whereby is generally between 4 to 16, preferably between 4 and 10, and more preferably between 4 and 7.

In the refining industry, the concept of petroleum fraction is well known to one skilled in the art and when the latter speaks of fraction C5, for example, he knows that said fraction necessarily will comprise several compounds with 6 carbon atoms and several other compounds with 4 carbon atoms.

A fraction can also be larger, for example, the C5-C6 fraction that for the most part contains the C5 and C6 compounds and a minor proportion of C4 and C7 compounds.

Within this text, we define the concept of petroleum fraction in the way that is familiar to one skilled in the art.

The invention applies particularly well to the fraction, in the separation, of the Cn Cn+1-type petroleum fractions, with n generally between 4 and 16, preferably between 4 and 10, and more preferably between 4 and 7.

The invention also applies to the fractionation, in the separation, of Cn-type petroleum fractions, with n generally between 4 and 16, preferably between 4 and 10, and more preferably between 4 and 7; these fractions are in particular the C4, C5, C6 and C7 fractions.

It was seen above that the object of the invention is to carry out the separation of certain molecules that are contained in a hydrocarbon feedstock such as a petroleum fraction. In particular, the invention allows the fractionation of a feedstock such as a petroleum fraction that has hydrocarbon molecules of close boiling points.

Inside of a given fraction, different chemical families can be encountered, and inside of the same family, elements of the same chemical formula but of different geometric configurations referred to as isomers can be encountered. The invention is therefore suitable for the separation of these isomers, but more particularly for the separation of a normal paraffin inside a hydrocarbon feedstock or any petroleum fraction that has in particular the same number of carbon atoms as the normal paraffin in question.

The installation according to the invention can comprise, in a manner known to one skilled in the art, pumps, valves, regulating devices and other streams in this type of installation and that are not, for the sake of simplification, described below and reproduced in the attached figures. Likewise, all recycling steps are possible for the streams obtained from the installation.

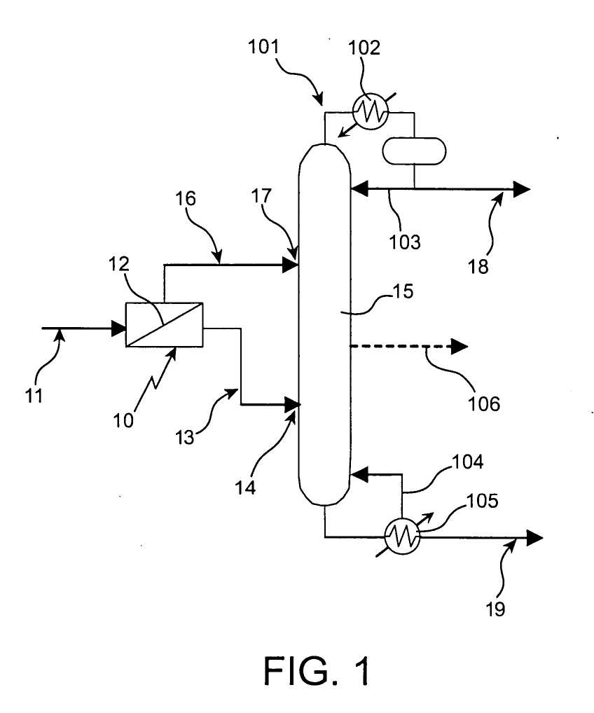

In a first configuration of the installation according to the invention (configuration 1), the membrane separation unit is located in such a way as to ensure pre-treatment of the feedstock of the distillation column, upstream from the latter; such a configuration is illustrated in FIG. 1.

The membrane separation unit (10) is actually fed by the flow or stream of feedstock to be treated that is routed via a conduit (11).

Inside of said membrane separation unit, there is at least one membrane (12) that allows a fraction of the feedstock (the permeate) to pass and that retains another fraction of said feedstock (retentate).

The flow or stream of permeate that is obtained from the membrane separation unit (10) is routed via a conduit (13) to the distillation column, and it is introduced at a certain point (14) of said distillation column (15).

The flow or stream of retentate exiting from the membrane separation unit is routed via a conduit (16) to the distillation column, and it is introduced at a point (17) of the column that is generally separate from the point (14) for introduction of the permeate.

The flow or stream of permeate routed via the conduit (13) generally will be enriched with normal paraffins, in the case where the membrane is selective for normal paraffins and can be introduced into the column, generally at a level (14) where the composition of said permeate is as close as possible to the composition of the flow or stream inside the column (15) at the level of said introduction.

In the same manner, the flow or stream of retentate that is routed via the conduit (16) will generally have reduced levels of normal paraffins in the case where the membrane is selective for normal paraffins and can be introduced into the column at a level (17) where the composition of said retentate (16) is as close as possible to the composition of the flow or stream that is inside the column (15) at the level of said introduction.

In some cases, based on the purity of the permeate (13) or the retentate (16), it will be possible to recover one or the other directly, without having to introduce it into the column (15), either to be sent into a storage unit or to be sent to a treatment unit, such as an isomerization or any other refinery process that can treat these streams.

The permeate, for example, is enriched with normal paraffins, while the retentate is enriched with branched paraffins comprising the same number of carbon atoms.

In a standard manner, a top flow or stream is drawn off from the column at its peak via the conduit (18), and a bottom or base flow or stream is drawn off from the column (15), at its base, via the conduit (19). In this entire description, for simplification, top flow or stream (18) is defined as the flow or stream that is obtained from the condensation in a condenser (102) of a flow or stream (101) removed at the peak, at the top, of the column (12) and that is not reintroduced directly into the column via the conduit (103). Likewise, by simplification here, bottom or base flow or stream (19) is defined as the flow or stream (19) that is obtained from the reboiler (105) and that is not directly reintroduced at the base, at the bottom of the column via the conduit (104).

It optionally is possible to draw off at least one lateral stream (106) from the column (15).

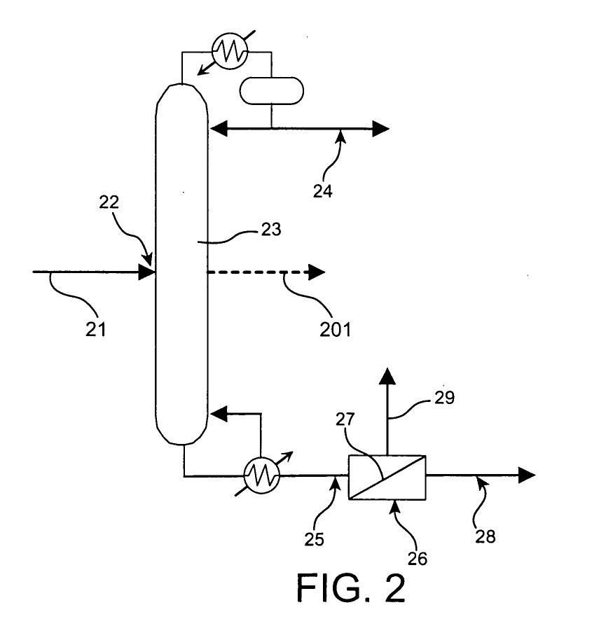

In a second configuration of the installation according to the invention (configuration 2), the membrane separation unit is located in such a way as to ensure post-treatment of the bottom flow or stream of the distillation column, downstream from the latter; such a configuration is illustrated in FIG. 2.

The flow or stream of feedstock, routed via a conduit (21), feeds at a point (22) the distillation column (23) that fractionates it into at least two flows or streams: namely a top flow or stream (24), a bottom or base flow or stream (25), and optionally one or more lateral streams (201).

The top flow or stream (24) is sent downstream or upstream from the separation installation, and the bottom flow or stream (25) feeds the membrane separation unit (26) that is equipped with at least one membrane (27).

The flow or stream of permeate (28) that is obtained from the membrane separation unit (26) can be sent upstream—i.e., a recycling of at least a portion of the stream or flow is carried out at any point of the installation—or downstream—i.e., a recovery of the stream or flow is carried out—from said separation unit.

The flow or stream of retentate (29) that is obtained from the membrane separation unit (26) can be sent upstream—i.e., a recycling of at least a portion of the stream or flow is carried out at any point—or downstream—i.e., a recovery of the stream or flow is carried out—from the separation unit.

Thus, in some cases, based on the purity of the retentate (29) or the permeate (28), it will be possible to reintroduce one or the other into the column (23) at a level where the composition of said permeate or retentate will be as close as possible to the composition of the flow or stream inside the column at the level of said reintroduction (or else these streams can be recovered).

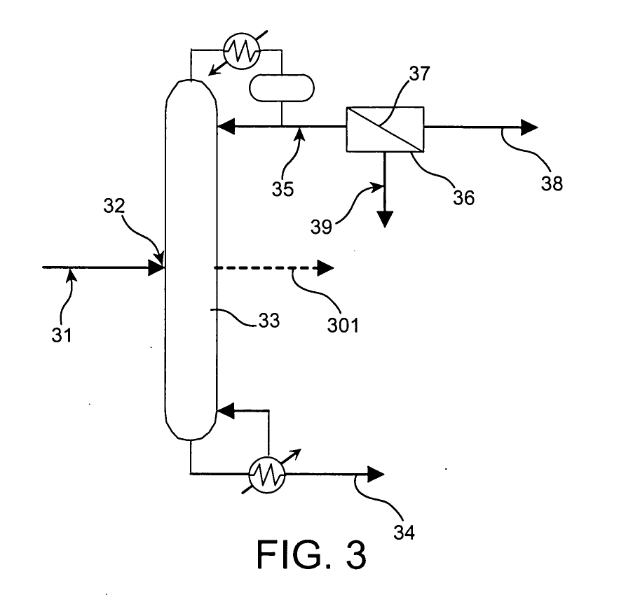

In a third configuration (configuration 3) of the installation according to the invention, the membrane separation unit is located in such a way as to ensure post-treatment of the top flow or stream of the distillation column, downstream from the latter; such a configuration is illustrated in FIG. 3.

The flow or stream of feedstock, routed via a conduit (31), feeds at a point (32) the distillation column (33) that fractionates it into at least two flows or streams: namely a bottom stream, a top stream, and optionally at least one lateral stream (301). The bottom flow or stream routed via the conduit (34) is recovered (according to FIG. 3), but it can also be recycled in part (not shown), and the top flow or stream (35) feeds the membrane separation unit (36) containing at least one membrane (37).

The flow of permeate (38) that is obtained from the membrane separation unit can be sent upstream or downstream (see the definition provided above) from the separation installation.

The flow of retentate (39) that is obtained from the membrane separation unit can be sent upstream or downstream from the separation installation.

In some cases, based on the purity of the retentate (39) or the permeate (38), it will be possible to reintroduce one or the other into the column (33) at a level where the composition of said permeate or retentate will be as close as possible to the composition of the flow or stream inside the column at the level of said reintroduction.

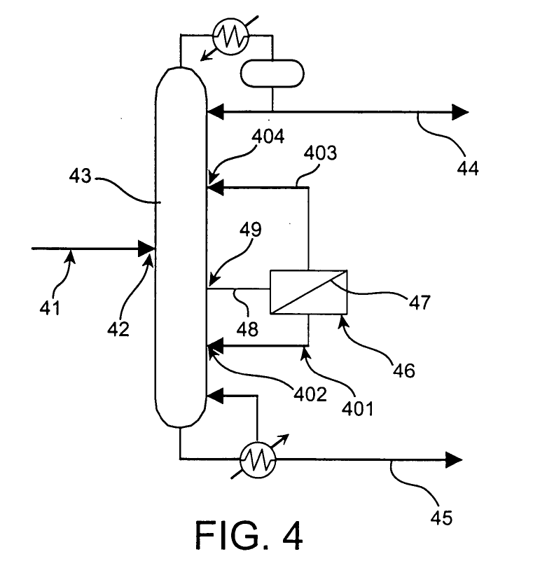

In a fourth configuration, (configuration 4) of the installation according to the invention, the membrane separation unit is located in such a way as to ensure the bypass in a lateral flow or stream from the distillation column, downstream from the latter: such a configuration is illustrated in FIG. 4.

The flow or stream of feedstock, routed via a conduit (41), feeds at a point (42) the distillation column (43) that fractionates it into at least two flows or streams. The top flow or stream that is routed via the conduit (44) is sent downstream or upstream from the separation installation, and the bottom flow or stream that is routed via the conduit (45) is sent downstream or upstream from the installation.

The membrane separation unit (46), containing at least one membrane (47), is fed by a lateral flow or stream that is routed via a conduit (48) that is obtained from the column (43) at a point (49).

The permeate stream that is routed via a conduit (401) and that is enriched with, for example, normal paraffins, if the membrane is selective for normal paraffins, is generally reintroduced in the column at a level (402) where its composition is the closest possible to the composition of the inside flow or stream of the column (43).

The retentate stream that is routed via the conduit (403) and that is low in, for example, normal paraffins, if the membrane is selective for normal paraffins, is generally reintroduced into the column at a level (404) where its composition is the closest possible to the composition of the inside flow or stream of the column.

In certain cases, the permeate and/or the retentate can be recovered directly without being reintroduced into the column.

The invention also comprises all the processes and installations that result from any combination of two or more of the basic configurations described above.

For example, it is possible to use two membrane separation units, one located in such a way as to ensure the pre-treatment configuration of the feedstock of the column, the other located in such a way as to ensure the post-treatment configuration of the bottom flow or stream of the column.

It is also possible to use two distillation columns or more.

The invention therefore comprises installations combining at least one distillation column and at least one membrane separation unit, whereby said membrane separation unit or units each comprise one or more membrane modules.

It is to account for these possibilities that the expression “at least one membrane separation unit” and the expression “at least one distillation column” are generally used in this text.

The advantage of the invention is to be assessed by comparison with the situation of a separation of different hydrocarbon molecules that belong to a petroleum fraction, in the previously defined meaning, carried out only with a column or a group of distillation columns.

For example, in the case of a separation of a normal paraffin within a petroleum fraction that contains said normal paraffin, the fact of adding a membrane separation unit in combination with the distillation column will be reflected by an economic gain:
For a given normal paraffin purity and a flow rate of the feedstock to be treated, the membrane separation unit will make it possible to increase the separation capacity of the column that will be employed, either by reducing the reflux rate or by reducing the number of plates, or by reducing the heat loads in the reboiler and/or the condenser.
For a given column and a fixed normal paraffin purity, the membrane separation unit will make it possible to treat a more significant feedstock flow rate. Actually, a distillation column is generally constructed to treat a given feedstock flow rate, and any increase in capacity will be reflected by an overconsumption of required heat loads in the reboiler and in the condenser, as well as by an increase of the traffic inside the column that has a hydrodynamic limit that is well known to one skilled in the art under the name of clogging.

In a distillation column, good operation depends for the most part on the upward flow conditions of the vapor and the downward flow conditions of the liquid, i.e., the co-existence of a gas flow and a liquid flow that are antagonists, together referred to as internal traffic.

If the flow rate of this internal traffic increases either by increasing the flow rate of the feedstock or by increasing the reflux rate, the speed of the vapor in the column will also increase, and beyond a certain value, the vapor flow will disrupt the downward circulation of the liquid flow.

If the traffic is increased again, the vapor flow can stop the flow of the liquid flow completely. There is no longer circulation in the column, therefore there is more exchange between liquid and vapor. The column no longer ensures separation and quickly fills up with liquid. This is the phenomenon of clogging.

This phenomenon is quantified by one skilled in the art by means of the clogging factor of the column.

This clogging factor takes into account the hydrodynamic properties of the liquid and vapor phases (density, viscosity, . . . ) as well as the passage sections that are available for the flow according to the geometry of the column and its internals.

The value of the clogging factor is expressed by fraction or by percentage.

This value very clearly suggests at what position the operating point of the column is located relative to the phenomenon of clogging.

In a general way, one skilled in the art selects a clogging factor not exceeding 0.8 (or else 80 according to whether it is expressed by fraction or by %).

In other words, one skilled in the art chooses to operate under conditions that are generally 20% below the clogging point of the column.

Beyond a clogging factor of 0.8 (or 80 expressed by percentage), the operation of the column remains possible but exhibits a risk to the extent that the vapor flow begins to disrupt the flow of the liquid phase that then becomes irregular. Such a situation is reflected by an unstable operation of the column, a loss of efficiency of the plates, and the production of products of degraded quality.

The introduction of the membrane separation unit will in some way absorb the increase of the feedstock to be treated by allowing the column to continue operating on the operating point for which it was sized, i.e., sufficiently below the clogging limit.

Finally, for a column and a fixed feedstock flow rate, the introduction of the membrane separation unit makes it possible to increase the separation capacity of said column and therefore to obtain a normal paraffin at a higher purity level.

Another advantage of the device according to the invention is the modularity that it makes it possible to introduce. Actually, a distillation column, for the reasons given above, cannot absorb a modification of the production targets (in terms of flow rate of the feedstock to be treated or purity of the top flow or bottom flow) without a significant modification, such as, for example, the increase of the number of plates.

In contrast, the introduction of a membrane separation unit by coupling with the column results in a much more flexible system than the column by itself, as will be demonstrated in the examples below.

In this invention, it is also conceivable to use a partition column instead of a simple distillation column. A detailed description of this type of partition column by Howard Rudd can be found in the supplement “The Chemical Engineer,” editor: “Institution of Chemical Engineers,” Davis Building, 165-171 Railway Terrace, Rugby, Warwickshire CV21 3HQ, Great Britain, dated Aug. 27, 1992. It is also possible to refer to the patent EP-1 205 460.

The coupling of a partition column with a membrane separation unit can be used in particular to separate the effluent of an isomerization reactor from a C7 hydrocarbon fraction.

Any type of membrane making it possible to make the separation between the linear paraffins and the branched paraffins, regardless of the organic membranes or polymers, inorganic, ceramic or mineral (at least partially composed of, for example, zeolite, silica, alumina, glass or carbon), or composites that consist of polymer and at least one inorganic compound (for example, the PDMS 1070 membrane of the Sulzer Chemtech Membrane Systems, Friedrichsthaler Strasse 19, D-66540 Neunkirchen, Germany) can be used within the scope of this invention.

The membrane separation unit can operate in vapor permeation mode, in pervaporation mode, or in hyperfiltration mode.

### Mode of Operation by Vapor Permeation

In the case of vapor permeation, the mixture to be separated circulates in a vapor phase upstream from the membrane. The compounds that constitute the permeate pass through the separating membrane according to a sorption-diffusion mechanism. Whereby the transfer of material through the membrane is induced by a partial pressure difference of each compound between the upstream face (retentate compartment) and the downstream face (permeate compartment) of the membrane, the permeate is recovered in vapor phase downstream from the membrane at a pressure that is lower than that of the retentate.

The permeate is then recovered by condensation on one or more cold points.

Among the preferred membrane materials are found polymers, in particular and in a non-exhaustive manner thermoplastic polymers, vitreous polymers or crystalline polymers, such as PPO (phenyl polyoxide) and derivatives, polyimides and polymer alloys employing polyimides, polyimides based on 6FDA-type dianhydrides (4,4′-(hexafluoroisopropylidene)diphthaleic acid) including the 6FDA-TrMP (polyimide obtained from the condensation of a 6FDA-type dianhydride and a trimethylphenyleneamine-type diamine) and 6FDA-TeMP (polyimide obtained from the condensation of a 6FDA-type dianhydric acid and a tetramethylphenyleneamine-type diamine), polyamides, aromatic polyamides, polyether imides, polyvinyl pyrrolidones, polycarbonates, polysulfones, cellulose and derivatives, or mixtures of polymers or copolymers employing at least one of the cited polymers.

The inorganic membranes with a molecular sieve base also have very advantageous performance levels for the n/isoparaffin separation.

In particular, numerous works of literature refer to MFI-type zeolitic-film-based membranes that make it possible to separate in a very effective manner the linear paraffins from the branched paraffins thanks to a diffusional selectivity mechanism.

It is also possible to consider using LTA-structural-type zeolite-based membranes, a zeolite that has a very good shape selectivity with regard to normal paraffins.

### Pervaporation Mode of Operation

In the case of pervaporation, the mixture to be separated circulates in liquid phase upstream from the membrane. The permeate passes through the membrane with a sorption-diffusion mechanism and vaporizes at the level of the phase downstream from the membrane. This vaporization is due to a lowering of the partial pressure of different components of the permeate, induced either by putting the compartment downstream from the membrane under vacuum or by flushing the downstream face of the membrane with a cover gas. The permeate is then recovered by condensation on one or more cold points.

### Hyperfiltration Mode of Operation

Another preferred embodiment of the membrane separation stage relates to hyperfiltration. Contrary to the vapor permeation or the pervaporation, the flow of material through the membrane is induced by a mechanical pressure difference between the two faces of the membrane (generally between 150 and 5 bar, preferably between 10 and 80 bar, and in a preferred manner between 10 and 70 bar). This membrane separation technique offers the advantage of being able to be operated in a relatively small temperature range (less than 150° C., preferably 100° C.) that is compatible with the conventional use of columns dedicated to the light n/iso paraffin separation (carbon number that is less than or equal to 8). Among the preferred materials that constitute the separating layer of membranes intended to carry out an n/iso paraffin separation by hyperfiltration, it is possible to cite in a non-exhaustive manner the polymers that may or may not have molecular sieving properties such as the polyimides (manufacturer: Grace Davison), the polyamides (manufacturers: Dow, Nitto, Toray), the polyether sulfones, the cellulose and derivatives (manufacturer: Hoechst), and the sulfonated polysulfones (manufacturer Celgard, Koch).

The invention will now be described with reference to the following examples, given by way of illustration and not limiting.

### EXAMPLES

The three examples below are intended to illustrate the advantages provided by the invention in three different configurations or embodiments of the process and the installation that are the object of the latter. In each of Examples 1, 2 and 3, Examples 1A, 1B; 2A, 2B; and 3A, 3B, in which only one distillation column is used, are reference examples, for comparison, which illustrate the prior art, while Examples 1C, 2C and 3C, in which a distillation column and a membrane separation unit are combined, are examples that illustrate the process and the installation according to the invention.
In a first configuration, according to the invention, the membrane separation unit is located so as to carry out a pre-treatment of the feedstock of the column: this configuration is illustrated by Example 1C, in which the membrane separation unit uses a membrane with an MFI-type layer;
In a second configuration, according to the invention, the membrane separation unit is located so as to carry out a treatment of a lateral flow of the column: this configuration is illustrated by Example 2C in which the membrane separation unit uses a membrane with an MFI-type layer;
In a third configuration, according to the invention, the membrane separation unit is located so as to carry out a treatment of a lateral flow of the column: this configuration is illustrated by Example 3C, in which the membrane separation unit employs a membrane with a polyimide film.

### Example 1

### Membrane Separation Unit Located Upstream from the Column

This Example 1 makes it possible to illustrate the advantages of the separation process according to the invention, when the membrane separation unit is located in such a way as to ensure pre-treatment of the feedstock that feeds the distillation column.

This example was carried out by means of a digital simulation by using the PRO2 software marketed by the SIMSCI Company (Invensys Systems, Inc./SimSci-Esscor—26561 Rancho Parkway South—Lake Forest, Calif. 92630 USA).

The example relates to the separation of isobutane and normal butane in a mixture that contains primarily normal butane and isobutane in similar proportions (50/50) as well as traces of compounds with 3 and 5 carbon atoms.

The feedstock feeds a distillation column at the 44^{th} theoretical plate by numbering the plates from top to bottom starting from the top plate of the column.

The column consists of 92 theoretical plates and operates at between 0.8 and 1.0 MPa of absolute pressure with a temperature of 50° C. in the condenser.

The top product of the column consists of pure isobutane with 98.2% by mass with 0.7% of normal butane and 1.1% by mass of propane.

The column is equipped with a lateral draw-off located at 12 theoretical plates above the bottom plate of the column. This lateral draw-off consists of pure normal butane with 95.8% by mass with 4.1% by mass of isobutane and 0.2 of isopentane.

Finally, the bottom product consists of 58.0% by mass of normal butane, 41.9% by mass of isopentane and 0.1% by mass of isobutane.

### Example 1 is divided into three examples

### Examples 1A, 1B and 1C

1A: This is a reference example, for comparison, according to the prior art, in which a single column that operates at 100% of its nominal capacity is used;

1B: This is a second example, for comparison, according to the prior art, in which the same column is used as in the Reference Example 1A, for comparison, but this column operates at a flow rate that is increased by 35% by mass relative to Example 1A by maintaining the same purities of products;

1C: This is an example according to this invention in which the same column is used as in the Reference Example 1A, for comparison, and as in Example 1B, operating in line with Example 1B at 35% additional capacity. According to the invention, however, this column is coupled to a membrane module that carries out a pre-treatment of the feedstock.

The permeate and the retentate that are obtained are introduced into the column, respectively, at theoretical plates Nos. 71 and 21.

The membrane is operated in “vapor permeation” mode at a pressure of 1.2 MPa and at a temperature of 220° C.

The membrane consists of a selective layer of MFI-type zeolites, with a thickness that is equivalent to 15 μm deposited on a porous multitubular substrate of alpha-alumina.

The embodiment of the installation according to the invention that is used in Example 1C is the one that is illustrated by FIG. 1, with a lateral draw-off.

The material balances as well as the primary characteristics of the flows or streams for each of Examples 1A, 1B and 1C are provided in summary tables 1A, 1B, and 1C and in summary table 1D below:

TABLE 1A
Column without a Membrane (Example 1A)

| Composition in | | Top | Lateral Draw- | |
|---|---|---|---|---|
| % by Mass | Feedstock | Product | off | Bottom Product |
| Propane | 0.5 | 1.1 | 0.0 | 0.0 |
| Isobutane | 45.0 | 98.2 | 4.1 | 0.1 |
| Normal Butane | 54.0 | 0.7 | 95.8 | 58.0 |
| Isopentane | 0.5 | 0.0 | 0.2 | 41.9 |
| Total Flow | 10,000 | 4,355 | 5551 | 93 |
| Rate in kg/h | | | | |

TABLE 1B
Column without a Membrane with an Increase of 35% by
Mass of the Amount of Feedstock (Example 1B)

| Composition in | | Top | Lateral | |
|---|---|---|---|---|
| % By Mass | Feedstock + 35% | Product | Draw-off | Bottom Product |
| Propane | 0.5 | 1.1 | 0.0 | 0.0 |
| Isobutane | 45.0 | 98.2 | 4.1 | 0.1 |
| Normal Butane | 54.0 | 0.7 | 95.8 | 58.0 |
| Isopentane | 0.5 | 0.0 | 0.2 | 41.9 |
| Total Flow | 13,500 | 5,880 | 7,494 | 126 |
| Rate in kg/h | | | | |

TABLE 1C
Column with a Membrane with an Increase of 35%
by Mass of the Amount of Feedstock (Example 1c)

| | | | | | Lat- | Bot- |
|---|---|---|---|---|---|---|
| Composition | | | | Top | eral | tom |
| in % | Membrane | Per- | | Prod- | Draw | Prod- |
| By Mass | Feedstock | meate | Retentate | uct | -off | uct |
| Propane | 0.5 | 0.9 | 0.0 | 1.1 | 0.0 | 0.0 |
| Isobutane | 45.0 | 14.7 | 81.1 | 98.2 | 4.0 | 0.1 |
| Normal | 54.0 | 84.4 | 17.8 | 0.7 | 95.7 | 59.6 |
| Butane | | | | | | |
| Isopentane | 0.5 | 0.0 | 1.1 | 0.0 | 0.2 | 40.3 |
| Total Flow | 13,500 | 7,345 | 6,155 | 5,880 | 7,498 | 122 |
| Rate in kg/h | | | | | | |

TABLE 1D
FOR COMPARISON

| | Number of | Maximum | Heat Load | Heat Load |
|---|---|---|---|---|
| | Theoretical | Clogging | of the | of the |
| | Plates | Factor | Condenser | Reboiler |
| Single Column | 92 | 80.5 | 2.9 MM kcal | 3.1 MMk cal |
| Single Column | 92 | 121.9 | 3.9 MM kcal | 4.1 MM kcal |
| with 35% | | | | |
| Capacity | | | | |
| Increase | | | | |
| Column + | 92 | 72.9 | 2.9 MM kcal | 3.1 MM kcal |
| Membrane | | | | |
| with | | | | |
| 35% Capacity | | | | |
| Increase | | | | |

Example 1B corresponds to the single column that operates with an increase of 35% of the feedstock to be treated by maintaining the same performance levels of separation. This Example 1 B cannot be implemented in practice for two reasons:
1) The heat loads of the reboiler and the condenser increase respectively by 32.2% and 34.4%; however, the corresponding utilities, cooling water and low-pressure vapor, are not always available on the production site.
2) The clogging factor exceeds 120, which means a virtually total clogging of the column that becomes inoperable and a drastic fall in performance levels.

The introduction of the membrane module in pre-treatment makes it possible to increase by 35% the flow rate of the feedstock without consequence on the initial heat loads and with a clogging factor of 72.9 that is actually better than in the basic case, which is quite remarkable.

### Example 2

### Membrane Separation Unit Located in Such a Way as to Ensure Treatment of a Lateral Flow or Stream of the Column

This example makes it possible to illustrate the advantages of the separation process according to the invention, when the membrane separation unit is located in such a way as to ensure treatment of a lateral flow or stream of the distillation column.

This example was carried out essentially by a digital simulation by means of PRO2 software marketed by the SIMSCI Company (Invensys Systems, Inc./SimSci-Esscor—26561 Rancho Parkway South—Lake Forest, Calif. 92630 USA).

The process that is illustrated by this example is dedicated to the separation of isopentane from a petroleum fraction that contains saturated hydrocarbons with 5 and 6 carbon atoms. The feedstock feeds a distillation column to the 29^{th} theoretical plate by numbering the plates from top to bottom starting from the top plate of the column. The column consists of 58 theoretical plates and operates between at 0.2 and 0.3 MPa of absolute pressure with a temperature of 61° C. in the condenser.

The top product of the column consists of pure isopentane with 93.7% by mass with 2.5% of normal pertane, the remainder consisting of normal butane and isobutane.

The bottom product consists of 44.9% by mass of normal pertane, 25.5% by mass of normal hexane and 1% by mass of isopentane, the remainder being molecules with 5 and 6 carbon atoms.

### Example 2 is divided into three examples

### Examples 2A 2B and 2C:

2A. This is a reference example, for comparison, according to the prior art, in which a single column that operates at 100% of its nominal capacity is used.

2B. This is a second example, for comparison, according to the prior art, in which the same column as in the Reference Example 2A, for comparison, is used, i.e., a column of the same diameter and having the same number of theoretical plates but operating with stricter purity specifications of the bottom and top products: 0.8% by mass of normal pentane in the top product instead of 2.5% by mass, and 0.5% by mass of isopentane in the bottom product instead of 1% by mass.

2C. This is an example according to this invention in which the same column as in the Reference Example 2B, for comparison, is used, treating the same feedstock flow rate and providing products with stricter specifications but with a membrane module that is located in parallel with the column. A liquid flow is removed at 30% of the theoretical plates above the bottom plate. The permeate and the retentate that are obtained are introduced into the column, respectively, with theoretical plates Nos. 53 and 27.

The membrane, operated in “vapor permeation” mode, at a temperature of 300° C., consists of a selective layer of MFI-type zeolites with an equivalent thickness of 15 μm deposited on a porous multitubular substrate of alpha-alumina.

The embodiment of the installation according to the invention that is used in Example 2C is illustrated by FIG. 4.

The material balances as well as the primary characteristics of the flows or streams for each of Examples 2A, 2B and 2C are provided in the summary tables 2A, 2B and 2C below, and the clogging factors and heat loads of the condenser and the reboiler for each of the examples are compared in Table 2D.

TABLE 2A
Column Without a Membrane (Example 2A)

| Composition in % | | Top Product, 2.5% | Bottom Product, |
|---|---|---|---|
| by Mass | Feedstock | Pentane | 1% Isopentane |
| IBUTANE | 1.0 | 1.9 | 0.0 |
| BUTANE | 1.0 | 1.9 | 0.0 |
| IPENTANE | 50.0 | 93.7 | 1.0 |
| CP | 0.5 | 0.0 | 1.1 |
| 22MB | 4.0 | 0.0 | 8.5 |
| 23MB | 1.0 | 0.0 | 2.1 |
| MCP | 1.0 | 0.0 | 2.1 |
| CH | 1.0 | 0.0 | 2.1 |
| PENTANE | 22.5 | 2.5 | 44.9 |
| 2MP | 4.0 | 0.0 | 8.5 |
| 3MP | 2.0 | 0.0 | 4.2 |
| HEXANE | 12.0 | 0.0 | 25.5 |
| Total Flow Rate in | 10,000 | 5,285 | 4,715 |
| kg/h | | | |

TABLE 2B
Column Without a Membrane and Purer Products (Example 2B)

| Composition in % | | Top Product, 0.8% | Bottom Product, |
|---|---|---|---|
| by Mass | Feedstock | Pentane | 0.5% Isopentane |
| IBUTANE | 1.0 | 1.9 | 0.0 |
| BUTANE | 1.0 | 1.9 | 0.0 |
| IPENTANE | 50.0 | 95.4 | 0.5 |
| CP | 0.5 | 0.0 | 1.0 |
| 22MB | 4.0 | 0.0 | 8.4 |
| 23MB | 1.0 | 0.0 | 2.1 |
| MCP | 1.0 | 0.0 | 2.1 |
| CH | 1.0 | 0.0 | 2.1 |
| PENTANE | 22.5 | 0.8 | 46.2 |
| 2MP | 4.0 | 0.0 | 8.4 |
| 3MP | 2.0 | 0.0 | 4.2 |
| HEXANE | 12.0 | 0.0 | 25.1 |
| Total Flow Rate in | 10,000 | 5,218 | 4,782 |
| kg/h | | | |

TABLE 2C
Column With a Membrane and Purer Products (Example 2C)

| | | | | | | Bottom |
|---|---|---|---|---|---|---|
| | | Top | | | | Product, |
| Composition | | Product, | Membrane | | | 0.5% |
| in % by Mass | Feedstock | 0.8% Pentane | Feed | Permeate | Retentate | Isopentane |
| IBUTANE | 1.0 | 1.9 | 0.0 | 0.0 | 0.0 | 0.0 |
| BUTANE | 1.0 | 1.9 | 0.0 | 0.0 | 0.0 | 0.0 |
| IPENTANE | 50.0 | 95.4 | 31.2 | 2.7 | 69.0 | 0.5 |
| CP | 0.5 | 0.0 | 0.6 | 0.1 | 1.3 | 1.0 |
| 22MB | 4.0 | 0.0 | 3.0 | 0.3 | 6.6 | 8.4 |
| 23MB | 1.0 | 0.0 | 0.5 | 0.0 | 1.1 | 2.1 |
| MCP | 1.0 | 0.0 | 0.4 | 0.0 | 0.9 | 2.1 |
| CH | 1.0 | 0.0 | 0.4 | 0.0 | 0.8 | 2.1 |
| PENTANE | 22.5 | 0.8 | 57.6 | 90.9 | 13.4 | 46.2 |
| 2MP | 4.0 | 0.0 | 1.9 | 0.2 | 4.1 | 8.4 |
| 3MP | 2.0 | 0.0 | 0.9 | 0.1 | 1.9 | 4.2 |
| HEXANE | 12.0 | 0.0 | 3.6 | 5.7 | 0.8 | 25.1 |
| Total Flow | 10,000 | 5,218 | 8,500 | 4,847 | 3,653 | 4,782 |
| Rate in kg/h | | | | | | |

TABLE 2D
FOR COMPARISON

| | Maximum | | |
|---|---|---|---|
| | Clogging | Heat Load of | Heat Load of |
| | Factor | the Condenser | the Reboiler |
| Single Column | 79 | 4.2 MM kcal | 3.4 MM kcal |
| 2.5% by Mass | | | |
| of Pentane at | | | |
| the Top | | | |
| 1% by Mass of | | | |
| Isopentane at | | | |
| the Bottom | | | |
| Single Column | 140 | 6.5 MM kcal | 5.7 MM kcal |
| 0.8% by Mass | | | |
| of Pentane at | | | |
| the Top | | | |
| 0.5% by Mass | | | |
| of Isopentane at | | | |
| the Bottom | | | |
| Column + Membrane | 80 | 4.2 MM kcal | 3.4 MM kcal |
| 0.8% by Mass | | | |
| of Pentane at | | | |
| the Top | | | |
| 0.5% by Mass | | | |
| of Isopentane at | | | |
| the Bottom | | | |

It is noted, therefore, that with a single column, an increase of the strictness of specifications on the top and bottom products by maintaining the same separation performance levels is difficult for two reasons:
The heat loads of the reboiler and the condenser increase respectively by 55% and 68%, which represents too high an increase of the corresponding utilities
Then and primarily, the maximum clogging factor in the column exceeds 140, which means a strongly degraded operation with loss of performances and runs the risk of total clogging of the then totally inoperable column.

The introduction of the membrane module in pre-treatment makes it possible to increase the strictness of the specifications on top and bottom products without consequence on the heat loads in the reboiler and in the condenser and with a clogging factor of 79 that is equal to that of the basic case.

### Example 3

### Hyperfiltration Membrane in Treatment of a Lateral Flow of the Column

This example makes it possible to illustrate the advantages of the process of separation according to the invention when the membrane separation unit is located in such a way as to ensure treatment of a lateral flow of the distillation column.

This example was carried out essentially by a digital simulation by means of PRO2 software marketed by the SIMSCI Company (Invensys Systems, Inc./SimSci-Esscor—26561 Rancho Parkway South—Lake Forest, Calif. 92630 USA).

The example relates to the separation of isopentane from a petroleum fraction that contains saturated hydrocarbons with 5 and 6 carbon atoms. The feedstock feeds a distillation column at the 29th theoretical plate by numbering the plates from top to bottom starting from the top plate of the column. The column consists of 58 theoretical plates and operates at between 0.2 and 0.3 MPa of absolute pressure with a temperature of 61° C. in the condenser.

The top product of the column consists of pure isopentane with 93.7% by mass with 2.5% of normal pentane, whereby the remainder consists of normal butane and isobutane.

The bottom product consists of 44.9% by mass of normal pertane, 25.5% by mass of normal hexane and 1% by mass of isopentane, whereby the remainder consists of molecules with 5 and 6 carbon atoms.

### Example 3 is divided into three examples

### Examples 3A, 3B and 3C:

3A. This is a reference example, for comparison, according to the prior art, in which a single column that operates at 100% of its nominal capacity is used.

3B. This is a second example, for comparison, according to the prior art, in which the same column as in the Reference Example 3A, for comparison, is used, i.e., a column of the same diameter and that has the same number of theoretical plates, but operates with stricter purity specifications of the bottom and top products: 1.1% by mass of normal pentane in the top product instead of 2.5% by mass, and 0.8% by mass of isopentane in the bottom product instead of 1% by mass.

3C. This is an example according to this invention in which the same column is used as in the Reference Example 3B, for comparison, treating the same feedstock flow rate and providing the products with stricter specifications, but with a membrane module that is located in parallel with the column.

The membrane consists of a non-woven substrate on which is deposited a Lenzig P84 commercial-type polyimide film (HP Polymers, Lewisville, Tex. USA).

It is operated in hyperfiltration mode at a temperature of 80° C., at a retentate pressure that is equal to 60 bar and at a permeate pressure that is equal to 15 bar. A liquid flow is removed at 30% of the theoretical plates above the bottom plate.

The permeate and retentate that are obtained are introduced into the column, respectively, at 7% and 52% of the theoretical plates above the bottom plate.

The embodiment of the installation according to the invention that is used in Example 3C is illustrated by FIG. 4.

The material balances as well as the primary characteristics of the flows or streams for each of Examples 3A, 3B, and 3C are given in the summary tables 3A, 3B and 3C below, and the clogging factors and heat loads of the condenser and reboiler for each of the examples are compared in Table 3D.

TABLE 3A
Column Without a Membrane (Example 3A)

| Composition in % | | Top Product, 2.5% | Bottom Product, |
|---|---|---|---|
| by Mass | Feedstock | Pentane | 1% Isopentane |
| IBUTANE | 1.0 | 1.9 | 0.0 |
| BUTANE | 1.0 | 1.9 | 0.0 |
| IPENTANE | 50.0 | 93.7 | 1.0 |
| CP | 0.5 | 0.0 | 1.1 |
| 22MB | 4.0 | 0.0 | 8.5 |
| 23MB | 1.0 | 0.0 | 2.1 |
| MCP | 1.0 | 0.0 | 2.1 |
| CH | 1.0 | 0.0 | 2.1 |
| PENTANE | 22.5 | 2.5 | 44.9 |
| 2MP | 4.0 | 0.0 | 8.5 |
| 3MP | 2.0 | 0.0 | 4.2 |
| HEXANE | 12.0 | 0.0 | 25.5 |
| Total Flow Rate in | 10,000 | 5,285 | 4,715 |
| kg/h | | | |

TABLE 3B
Column Without a Membrane
with Purer Top and Bottom Products (Example 3B)

| Composition in % | | Top Product, 1.1% | Bottom Product, |
|---|---|---|---|
| by Mass | Feedstock | Pentane | 0.8% Isopentane |
| IBUTANE | 1.0 | 1.9 | 0.0 |
| BUTANE | 1.0 | 1.9 | 0.0 |
| IPENTANE | 50.0 | 95.1 | 0.8 |
| CP | 0.5 | 0.0 | 1.0 |
| 22MB | 4.0 | 0.0 | 8.4 |
| 23MB | 1.0 | 0.0 | 2.1 |
| MCP | 1.0 | 0.0 | 2.1 |
| CH | 1.0 | 0.0 | 2.1 |
| PENTANE | 22.5 | 1.1 | 45.9 |
| 2MP | 4.0 | 0.0 | 8.4 |
| 3MP | 2.0 | 0.0 | 4.2 |
| HEXANE | 12.0 | 0.0 | 25.1 |
| Total Flow Rate in | 10,000 | 5,219 | 4,781 |
| kg/h | | | |

TABLE 3C
Column With a Membrane and Purer Top and Bottom Products (Example 3C)

| | | | | | | Bottom |
|---|---|---|---|---|---|---|
| | | | | | | Product, |
| Composition | | Top Product, | Membrane | | | 0.8% |
| in % by Mass | Feedstock | 1.1% Pentane | Feed | Permeate | Retentate | Isopentane |
| IBUTANE | 1.0 | 1.9 | 0.0 | 0.0 | 0.0 | 0.0 |
| BUTANE | 1.0 | 1.9 | 0.0 | 0.0 | 0.0 | 0.0 |
| IPENTANE | 50.0 | 95.1 | 31.2 | 5.9 | 59.9 | 0.8 |
| CP | 0.5 | 0.0 | 0.5 | 0.1 | 1.0 | 1.0 |
| 22MB | 4.0 | 0.0 | 2.8 | 0.5 | 5.4 | 8.4 |
| 23MB | 1.0 | 0.0 | 0.5 | 0.1 | 0.9 | 2.1 |
| MCP | 1.0 | 0.0 | 0.4 | 0.1 | 0.7 | 2.1 |
| CH | 1.0 | 0.0 | 0.4 | 0.1 | 0.7 | 2.1 |
| PENTANE | 22.5 | 1.1 | 57.9 | 87.2 | 24.7 | 45.9 |
| 2MP | 4.0 | 0.0 | 1.8 | 0.3 | 3.5 | 8.4 |
| 3MP | 2.0 | 0.0 | 0.8 | 0.2 | 1.6 | 4.2 |
| HEXANE | 12.0 | 0.0 | 3.7 | 5.5 | 1.6 | 25.1 |
| Total Flow | 10,000 | 5,219 | 8,500 | 4,514 | 3,986 | 4,781 |
| Rate in kg/h | | | | | | |

TABLE 3D
FOR COMPARISON

| | Maximum | | |
|---|---|---|---|
| | Clogging | Heat Load of | Heat Load of |
| | Factor | the Condenser | the Reboiler |
| Single Column | 79 | 4.2 MM kcal | 3.4 MM kcal |
| 2.5% by Mass | | | |
| of Pentane at | | | |
| the Top | | | |
| 1% by Mass of | | | |
| Isopentane at | | | |
| the Bottom | | | |
| Single Column | 104 | 5.2 MM kcal | 4.4 MM kcal |
| 0.8% by Mass | | | |
| of Pentane at | | | |
| the Top | | | |
| 0.5% by Mass | | | |
| of Isopentane at | | | |
| the Bottom | | | |
| Column + Membrane | 79 | 4.2 MM kcal | 3.4 MM kcal |
| 0.8% by Mass | | | |
| of Pentane at | | | |
| the Top | | | |
| 0.5% by Mass | | | |
| of Isopentane at | | | |
| the Bottom | | | |

With a single column, an increase of the strictness of specifications on the top and bottom products by maintaining the same separation performance levels is difficult for two reasons:
1) The heat loads of the reboiler and the condenser increase respectively by 24% and 29%, which represents too high an increase of the corresponding utilities.
2) The maximum clogging factor in the column exceeds 104, which means a strongly degraded operation with loss of performances and runs the risk of total clogging of the column that would then become totally inoperable.

The introduction of the membrane module in pre-treatment makes it possible to increase the strictness of the specifications on top and bottom products without consequence on the heat loads in the reboiler and in the condenser and with a clogging factor of 79 that is equal to that of the basic case, which is noteworthy.

The entire disclosures of all applications, patents and publications, cited herein and of corresponding French application No. 05/51,492, filed June 3, 2005 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for separating one or more compounds, including at least one n-paraffin, in a hydrocarbon feedstock, in which:
a) A stream, or a portion of a stream from said feedstock, is passed successively and in any order into at least one distillation column and at least one membrane separation unit comprising a selective membrane with regard to at least one n-paraffin; and
b) One or more stream(s) is (are) recovered from the column and the membrane separation unit, each of said streams being enriched respectively with one of said compounds, and at least one of said streams being enriched with at least one n-paraffin.

2. A process according to claim 1 for separating one or more compound(s), including at least one n-paraffin, in a hydrocarbon feedstock, in which:
c) A stream or a portion of a stream of said feedstock is passed into a distillation column in which at least one incoming stream enters and from which at least one outgoing stream exits, and in at least one membrane separation unit comprising a membrane that is selective with regard to at least one n-paraffin, a membrane separation unit being placed in the stream or streams entering and exiting from the distillation column;
d) One or more streams are recovered from the column and from the membrane separation unit or units, each of said streams being enriched respectively with one of said compounds and at least one of said streams being enriched with at least one n-paraffin.

3. A process according to claim 1, in which at least one of the streams obtained from the process is recycled completely or partially to the distillation column or columns and/or to the membrane separation unit or units and/or in the feedstock.

4. A process according to claim 1, in which the feedstock stream first is passed into a membrane separation unit that separates the feedstock stream into a retentate stream and a permeate stream.

5. A process according to claim 4, in which the retentate stream and the permeate stream are both introduced into the distillation column.

6. A process according to claim 4, in which one of the streams from among the permeate stream and the retentate stream is introduced into the column, and the other of these streams is recovered.

7. A process according to claim 5, in which the retentate stream and the permeate stream are introduced into the distillation column, at separate points, whereby the permeate stream is introduced into the distillation column at one point where the composition of an inside stream that travels through the column is close to the composition of the permeate stream and/or whereby the retentate stream is introduced into the distillation column at a point where the composition of an inside stream that travels through the distillation column is close to the composition of the retentate stream.

8. A process according to claim 4, in which a top stream, a bottom stream, and optionally at least one lateral stream are drawn off from the distillation column and recovered or recycled completely or partially.

9. A process according to claim 8, in which the top stream, the bottom stream and the possible lateral stream are recycled completely or partially to the distillation column and/or to the membrane separation unit, and/or to the feedstock stream.

10. A process according to claim 1, in which the feedstock stream first is passed into the distillation column that separates the stream of the feedstock into a top stream of the column, a bottom stream of the column, and optionally at least a lateral stream, at least one among the top stream, the bottom stream and the possible lateral stream or streams then being sent into a membrane separation unit.

11. A process according to claim 10, in which the top stream of the column is sent into a membrane separation unit that separates the top stream into a retentate stream and a permeate stream.

12. A process according to claim 10, in which the base stream of the column is sent into a membrane separation unit that separates the base stream into a retentate stream and a permeate stream.

13. A process according to claim 10, in which at least one lateral stream of the distillation column is sent into a membrane separation unit that separates the lateral stream into a retentate stream and a permeate stream.

14. A process according to claim 10, in which the permeate stream and the retentate stream are recovered or recycled completely or partially.

15. A process according to claim 14, in which the permeate stream and the retentate stream are both recovered.

16. A process according to claim 14, in which one of the streams from among the permeate stream and the retentate stream is at least partially recycled.

17. A process according to claim 14, in which the permeate and retentate streams are both partially recycled.

18. A process according to claim 14, in which the permeate stream and/or the retentate stream is/are recycled to the distillation column.

19. A process according to claim 18, in which the retentate stream and the permeate stream are both introduced again into the distillation column, at separate points, the permeate stream being introduced again into the distillation column at a point where the composition of an inside stream that travels through the column is close to the composition of the permeate stream, and/or the retentate stream is introduced again into the distillation column at a point where the composition of an inside stream that travels through the distillation column is close to the composition of the retentate stream.

20. A process according to claim 1, in which the membrane separation unit comprises one or more membrane modules.

21. A process according to claim 1, in which the feedstock stream is passed into several membrane separation units.

22. A process according to claim 21, in which the feedstock stream passes into two membrane separation units, one of which is placed in a stream entering the distillation column, and the other is placed in a stream exiting the distillation column that is selected from among the top stream of the column, the bottom stream of the column, and a lateral stream of the column.

23. A process according to claim 1, in which said hydrocarbon feedstock is selected from among the petroleum fractions Cn, where n is an integer from 4 to 16, preferably 4 to 10, and more preferably 4 to 7, whereby n can take on all the values from 4 to 16.

24. A process according to claim 1, in which said hydrocarbon feedstock is selected from among the petroleum fractions Cn—Cn +1 where n is an integer from 4 to 16, preferably 4 to 10, and more preferably 4 to 7, whereby n can take on all the values from 4 to 16.

25. A process according to claim 1, in which the compound or compounds to be separated are hydrocarbon compounds that have close boiling points.

26. A process according to claim 1, in which said compound or compounds to be separated are selected from among—in addition to the normal paraffins—the branched paraffins, the naphthenic compounds, the aromatic compounds and the olefinic compounds.

27. A process according to claim 1, in which said feedstock contains said normal paraffin and hydrocarbon compounds that have the same number of carbon atoms as said normal paraffin.

28. A process according to claim 27, in which the normal butane is separated from the isobutane contained in a hydrocarbon feedstock.

29. A process according to claim 27, in which the isopentane is separated from the normal pentane contained in a hydrocarbon feedstock.

30. A process according to claim 26, in which a C6 hydrocarbon fraction is fractionated into three streams, respectively enriched with naphthenic compounds, normal hexane, and branched paraffins.

31. A process according to claim 26, in which a C6 hydrocarbon fraction is fractionated into four streams, respectively enriched with naphthenic compounds, normal hexane, monobranched paraffins, and dibranched paraffins.

32. A process according to claim 26, in which a C7 hydrocarbon fraction is fractionated into three streams, respectively enriched with naphthenic compounds, normal heptane, and branched paraffins.

33. A process according to claim 25, in which a C7 hydrocarbon fraction is fractionated into four streams, respectively enriched with naphthenic compounds, normal heptane, monobranched paraffins, and dibranched paraffins.

34. An installation for separating one or more compound(s), including at least one n-paraffin, in a hydrocarbon feedstock, comprising:
e) At least one distillation column;
f) At least one membrane separation unit comprising a selective membrane with regard to at least one n-paraffin;
g) Means for feeding said installation with a stream of said hydrocarbon feedstock;
h) Means for ensuring fluid communication for the distillation column or columns and the membrane separation unit or units and passing said stream of said hydrocarbon feedstock, or a portion of said stream, successively and in any order into the column or columns and the membrane separation unit or units; and
i) Means for recovering one or more stream(s) from the column or columns and the membrane separation unit or units, each of said streams being enriched respectively with one of said compounds, and at least one of said streams being enriched with at least one n-paraffin.

35. An installation according to claim 34, in which a membrane separation unit is placed upstream from the distillation column in at least one of the streams entering into the distillation column, whereby said installation comprises means for sending the feedstock stream or a portion of the latter into said membrane separation unit, means for sending the retentate and/or the permeate of the membrane separation unit into said distillation column, and/or means for recovering the retentate and/or the permeate from said membrane separation unit, and means for drawing off and optionally recovering a top stream, a base stream, and optionally at least one lateral stream from said column.

36. An installation according to claim 34, in which a membrane separation unit is placed downstream from the distillation column in at least one of the streams exiting from the distillation column.

37. An installation according to claim 36, comprising means for sending the feedstock stream or a portion of the latter into the distillation column, means for sending a top stream of the distillation column into the membrane separation unit, and means for drawing off and optionally recovering a base stream of the distillation column and optionally at least one lateral stream from said distillation column.

38. An installation according to claim 36, comprising means for sending the feedstock stream or a portion of the latter into the distillation column, means for sending at least one lateral stream of the distillation column into the membrane separation unit, and means for drawing off and optionally recovering a base stream of the distillation column and a top stream of the distillation column.

39. An installation according to claim 36, comprising means for sending the feedstock stream or a portion of the latter into the distillation column, means for sending a base stream of the distillation column into the membrane separation unit, and means for drawing off and optionally recovering a top stream of the column and optionally at least one lateral stream from the distillation column.

40. An installation according to claim 36, comprising means for recovering the retentate and/or the permeate from the membrane separation unit and/or means for sending the retentate and/or the permeate into the distillation column.

41. An installation according to claim 34, comprising several membrane separation units.

42. An installation according to claim 41, comprising two membrane separation units, one being placed upstream from the distillation column and the other being placed downstream from the distillation column.

43. An installation according to claim 34, comprising means for recycling any of the streams obtained from the distillation column or columns and the membrane separation unit or units to the distillation column or columns and/or to the membrane separation unit or units and/or to the feedstock stream.

44. An installation according to claim 34, in which the membrane of the membrane separation unit is made of a material that is selected from among the inorganic materials, the organic materials, and the composite materials that comprise several different materials that are selected from among the organic and inorganic materials.

45. An installation according to claim 45, in which the membrane is made of an inorganic material that optionally is deposited on a porous substrate, for example of mineral oxide such as alumina.

46. An installation according to claim 45, in which the membrane is made of a molecular sieve such as a zeolite that is selected from among, for example, the MFI-type zeolites and the LTA-type zeolites.

47. An installation according to claim 34, in which the membrane separation unit operates in a vapor permeation mode.

48. An installation according to claim 34, in which the membrane separation unit operates in a pervaporation mode.

49. An installation according to claim 34, in which the membrane separation unit operates in a hyperfiltration mode.

50. An installation according to claim 34, in which the distillation column is a partition column.
